(19) United States
(12) Patent Application Publication
(10) Pub. No.: US 2025/0221671 A1
(43) Pub. Date: Jul. 10, 2025

(54) PHYSIOLOGICAL STATE PREDICTION BASED ON ACOUSTIC DATA USING MACHINE LEARNING

(71) Applicant: Apple Inc., Cupertino, CA (US)

(72) Inventors: Vikramjit MITRA, Pleasanton, CA (US); Jingping NIE, Fort Lee, NJ (US); Erdrin AZEMI, San Mateo, CA (US)

(21) Appl. No.: 18/817,058

(22) Filed: Aug. 27, 2024

Related U.S. Application Data
(60) Provisional application No. 63/619,236, filed on Jan. 9, 2024.

(51) Int. Cl. A61B 5/00; A61B 5/024

(52) U.S. Cl. CPC A61B 5/7267; A61B 5/7275; A61B 5/7282; A61B 5/02405; A61B 2562/0204

(57) ABSTRACT

The subject technology provides physiological state prediction based on acoustic data using machine learning. An apparatus receives input data comprising acoustic signal information associated with a user. The apparatus extracts one or more acoustic features from the acoustic signal information. The apparatus produces a trained machine learning model by training a neural network to predict one or more physiological states of the user from the one or more acoustic features.

### CROSS-REFERENCE TO RELATED APPLICATION(S)

This application claims the benefit of U.S. Provisional Application Ser. No. 63/619,236, entitled “PHYSIOLOGICAL STATE PREDICTION BASED ON ACOUSTIC DATA USING MACHINE LEARNING,” and filed on Jan. 9, 2024, the disclosure of which is expressly incorporated by reference herein in its entirety.

### TECHNICAL FIELD

The present description generally relates to physiological state prediction based on acoustic data using machine learning.

### BACKGROUND

Various physiological parameters of a user can be measured and analyzed to estimate other physiological measures indicative of the user's physiological state. Computer hardware has been utilized to make improvements across different industry applications including applications used to assess and monitor physiological activities.

### BRIEF DESCRIPTION OF THE DRAWINGS

Certain features of the subject technology are set forth in the appended claims. However, for purpose of explanation, several embodiments of the subject technology are set forth in the following figures.

FIG. 1 illustrates an example network environment in accordance with one or more implementations.

FIG. 2A illustrates an example computing architecture for a system providing for physiological state prediction based on acoustic data using machine learning in accordance with one or more implementations.

FIG. 2B conceptually illustrates an example of data training preparation in accordance with one or more implementations.

FIG. 3 conceptually illustrates an example overview of physiological state prediction based on acoustic data using machine learning in accordance with one or more implementations.

FIG. 4 is a flow chart of an example process that may be performed for physiological state prediction based on acoustic data in accordance with one or more implementations.

FIGS. 5-7 illustrate different example computing architectures of a machine learning model for physiological state prediction based on acoustic data in accordance with one or more implementations.

FIGS. 8-11 are schematic diagrams illustrating different example machine learning models in accordance with one or more implementations.

FIG. 12 illustrates an electronic system with which one or more implementations of the subject technology may be implemented.

### DETAILED DESCRIPTION

The detailed description set forth below is intended as a description of various configurations of the subject technology and is not intended to represent the only configurations in which the subject technology can be practiced. The appended drawings are incorporated herein and constitute a part of the detailed description. The detailed description includes specific details for the purpose of providing a thorough understanding of the subject technology. However, the subject technology is not limited to the specific details set forth herein and can be practiced using one or more other implementations. In one or more implementations, structures and components are shown in block diagram form in order to avoid obscuring the concepts of the subject technology.

Machine learning has seen a significant rise in popularity in recent years due to the availability of training data, and advances in more powerful and efficient computing hardware. Machine learning may utilize models that are executed to provide predictions in particular applications.

In an era characterized by the integration of Artificial Intelligence of Things (AIoT) and Internet of Medical Things (IoMT) into daily life, the capability to non-invasively and precisely monitor heart rate across various situations enables individuals to make informed health-related decisions. Emerging commercial-off-the-shelf (CoTs) digital stethoscopes intended for customers and ongoing research initiatives aim to empower users in self-monitoring their cardiovascular activities.

The human heart contains a wealth of physiological information. The monitoring of heart rate offers essential insights into cardiovascular health, stress assessment, and overall well-being. Yet, the inherent complexity of real-world environments, combined with the omnipresence of ambient noise, presents a substantial challenge for accurately detecting heart rates from phonocardiogram (PCG) signals. Traditional signal processing approaches have faced limitations in managing this inherent noise, necessitating a paradigm shift toward innovative solutions within the subject technology.

In the domain of health monitoring, acoustic signals play a significant role in conveying important information, with heart sounds exemplifying this function by providing data such as heart rate and identifying cardiac anomalies like murmurs. The subject technology provides for estimating one or more physiological states such as a heart rate and heart rate variability using audio signals obtained through sensors. Within this field, model-driven approaches are employed in estimating heart rate from concise segments of heart sounds, utilizing a phonocardiogram (PCG) dataset.

The subject technology includes a machine-learned model capable of identifying heart conditions such as heart murmur and arrhythmia. Deep learning can autonomously discern complex patterns from data including discerning the complexities of noisy PCG signals. Through the utilization of convolutional and recurrent neural networks, deep learning methodologies harbor the potential to extract heart rate information from the multitude of background noises, resulting in a robust and precise detection process. For deriving heart rate predictions, a sliding window methodology may be employed to extract snippets of heart rate sounds, utilizing a diverse set of acoustic features (e.g., Mel spectrogram, Mel-frequency cepstral coefficients, Power Spectral Density, and Root Mean Square) to characterize these snippets. Within the subject technology, the superiority of a two-dimensional (2D) convolutional neural network (2dCNN) for heart rate prediction is utilized, achieving a mean absolute error (MAE) in a range of about 1.3 to about 2.4 (e.g., at about 1.312 beats-per-minute (bpm)). The impact of different feature combinations is also disclosed, demonstrating that utilizing all four features yields the most optimal results.

Additionally, embodiments of the heart rate prediction model can be expanded into a multi-task learning (MTL) framework within the subject technology. Specifically, the subject technology may include a multi-feature, multitask model, enabling the generation of advanced heart rate predictions utilizing digital audio signals captured through input devices, such as microphones, integrated into a digital stethoscope. This framework enables the simultaneous prediction of heart rate and murmurs. The machine learning model (e.g., 2dCNN-MTL) within the subject technology can attain a heart rate prediction accuracy exceeding about 95%, surpassing traditional models while maintaining the MAE in the range of about 1.3 to about 2.4 (e.g., below 1.636 bpm) in heart rate prediction.

The subject technology aims to accurately assess heart-related parameters by leveraging machine learning techniques and signal processing algorithms applied to audio data acquired through sensor-based systems. As such, the subject technology is directed towards addressing the need for efficient and precise heart rate prediction while detecting specific cardiac abnormalities through non-invasive audio signal analysis. The subject technology emphasizes the potential of model-driven approaches for heart rate prediction and heart murmur prediction.

Implementations of the subject technology improve the ability of a given electronic device to provide sensor-based, machine-learning generated feedback to a user (e.g., a user of the given electronic device). These benefits therefore are understood as improving the computing functionality of a given electronic device, such as an end user device which may generally have less computational and/or power resources available than, e.g., one or more cloud-based servers.

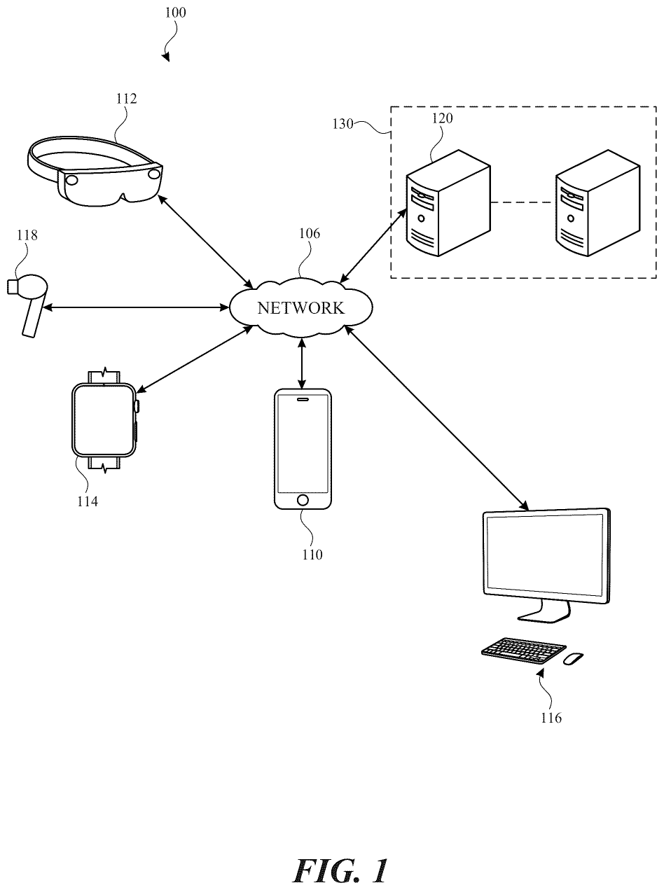

FIG. 1 illustrates an example network environment 100 in accordance with one or more implementations. Not all of the depicted components may be used in all implementations, however, and one or more implementations may include additional or different components than those shown in the figure. Variations in the arrangement and type of the components may be made without departing from the spirit or scope of the claims as set forth herein. Additional components, different components, or fewer components may be provided.

The network environment 100 includes an electronic device 110, an electronic device 112, an electronic device 114, an electronic device 116, an electronic device 118, a server 120, and a group of servers 130. The network 106 may communicatively (directly or indirectly) couple the electronic device 110 and/or the server 120. In one or more implementations, the network 106 may be an interconnected network of devices that may include, or may be communicatively coupled to, the Internet. For explanatory purposes, the network environment 100 is illustrated in FIG. 1 as including the electronic device 110, the electronic device 112, the electronic device 114, the electronic device 116, the electronic device 118, the server 120, and the group of servers 130; however, the network environment 100 may include any number of electronic devices and any number of servers or a data center including multiple servers.

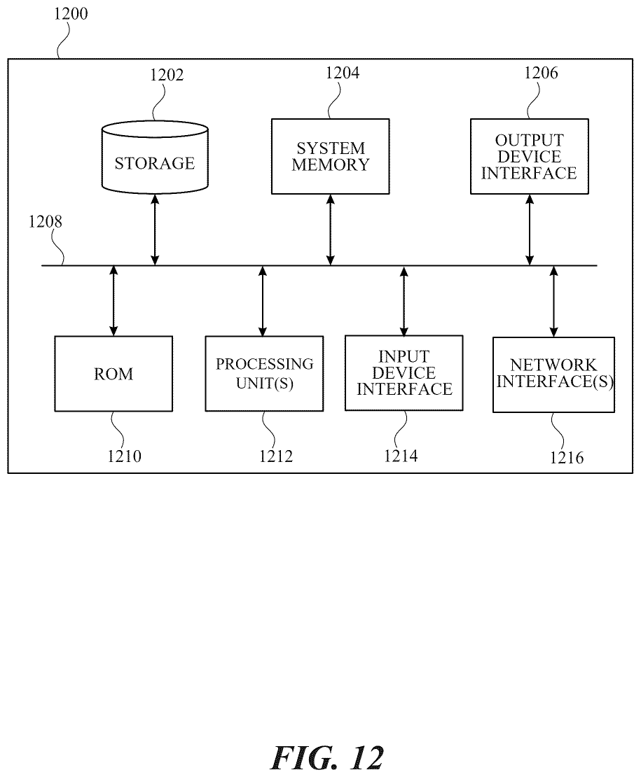

By way of example, the electronic device 110 is depicted as a mobile electronic device (e.g., smartphone). The electronic device 110 may be, for example, a desktop computer, a portable computing device such as a laptop computer, a peripheral device (e.g., a digital camera, headphones), a tablet device, a wearable device such as a watch, a band, and the like. The electronic device 110 may be, and/or may include all or part of, the electronic system discussed below with respect to FIG. 12.

By way of example, the electronic device 112 is depicted as a head mountable portable system that includes a display system capable of presenting a visualization of an extended reality environment to a user. The electronic device 112 may be, for example, a desktop computer, a portable computing device such as a laptop computer, a smartphone, a peripheral device (e.g., a digital camera, headphones), a tablet device, or a wearable device such as a watch. The electronic device 112 may be, and/or may include all or part of, the electronic system discussed below with respect to FIG. 12.

By way of example, the electronic device 114 is depicted as a watch. The electronic device 114 may be, for example, a desktop computer, a portable computing device such as a laptop computer, a smartphone, a peripheral device (e.g., a digital camera, headphones), or a tablet device. The electronic device 114 may be, and/or may include all or part of, the electronic system discussed below with respect to FIG. 12.

By way of example, the electronic device 116 is depicted as a desktop computer. The electronic device 116 may be, for example, a portable computing device such as a laptop computer, a smartphone, a peripheral device (e.g., a digital camera, headphones), a tablet device, a wearable device such as a watch, a band, and the like. The electronic device 116 may be, and/or may include all or part of, the electronic system discussed below with respect to FIG. 12.

By way of example, the electronic device 118 is depicted as an earbud. The electronic device 118 may be, for example, a desktop computer, a portable computing device such as a laptop computer, a smartphone, a peripheral device (e.g., a digital camera), a tablet device, a wearable device such as a watch, a band, and the like. The electronic device 118 may be, and/or may include all or part of, the electronic system discussed below with respect to FIG. 12.

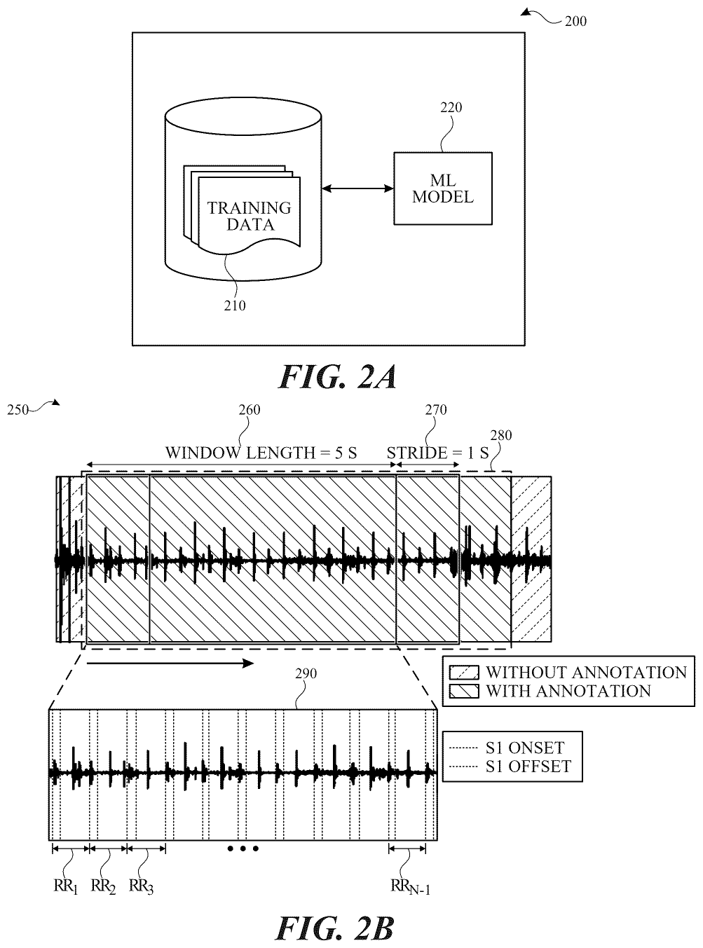

In one or more implementations, one or more of the electronic devices 110-130 may provide a system for training a machine learning model using training data, where the trained machine learning model is subsequently deployed to one or more of the electronic devices 110-130. One or more of the electronic devices 110-130 can collect data (e.g., health-related information) that is then used to train a machine learning model, which will be described with reference to FIGS. 2A-2B, 3 and 5-11. Further, one or more of the electronic devices 110-130 may provide one or more machine learning frameworks for training machine learning models and/or developing applications using such machine learning models. In an example, such machine learning frameworks can provide various machine learning algorithms and models for different problem domains in machine learning. In an example, the electronic device 110 may include a deployed machine learning model that provides an output of data corresponding to a prediction or some other type of machine learning output. In one or more implementations, training and inference operations that involve individually identifiable information of a user of one or more of the electronic devices 110-130 may be performed entirely on the electronic devices 110-130, to prevent exposure of individually identifiable data to devices and/or systems that are not authorized by the user.

The server 120 may form all or part of a network of computers or the group of servers 130, such as in a cloud computing or data center implementation. For example, the server 120 stores data and software, and includes specific hardware (e.g., processors, graphics processors and other specialized or custom processors) for rendering and generating content such as graphics, images, video, audio and multi-media files. In an implementation, the server 120 may function as a cloud storage server that stores any of the aforementioned content generated by the above-discussed devices and/or the server 120.

The server 120 and/or the group of servers 130 may provide a system for training a machine learning model using training data, where the trained machine learning model is subsequently deployed to the server 120, the group of servers 130 and/or to one or more of the electronic devices 110-118. In an implementation, the server 120 and/or the group of servers 130 may train a given machine learning model for deployment to a client electronic device (e.g., the electronic device 110, the electronic device 112, the electronic device 114, the electronic device 118). In one or more implementations, the server 120 and/or the group of servers 130 may train portions of the machine learning model that are trained using (e.g., anonymized) training data from a population of users, and one or more of the electronic devices 110-130 may train portions of the machine learning model that are trained using individual training data from the user of the electronic devices 110-118. The machine learning model deployed on the server 120, the group of servers 130 and/or one or more of the electronic devices 110-118 can then perform one or more machine learning algorithms. In an implementation, the server 120 and/or the group of servers 130 provides a cloud service that utilizes the trained machine learning model and/or continually learns over time.

In the example of FIG. 1, the electronic device 110 is depicted as a smartphone. However, it is appreciated that the electronic device 110 may be implemented as another type of device, such as a wearable device (e.g., a smart watch or other wearable device). The electronic device 110 may be a device of a user (e.g., the electronic device 110 may be associated with and/or logged into a user account for the user at a server). Although a single electronic device 110 is shown in FIG. 1, it is appreciated that the network environment 100 may include more than one electronic device, including more than one electronic device of a user and/or one or more other electronic devices of one or more other users.

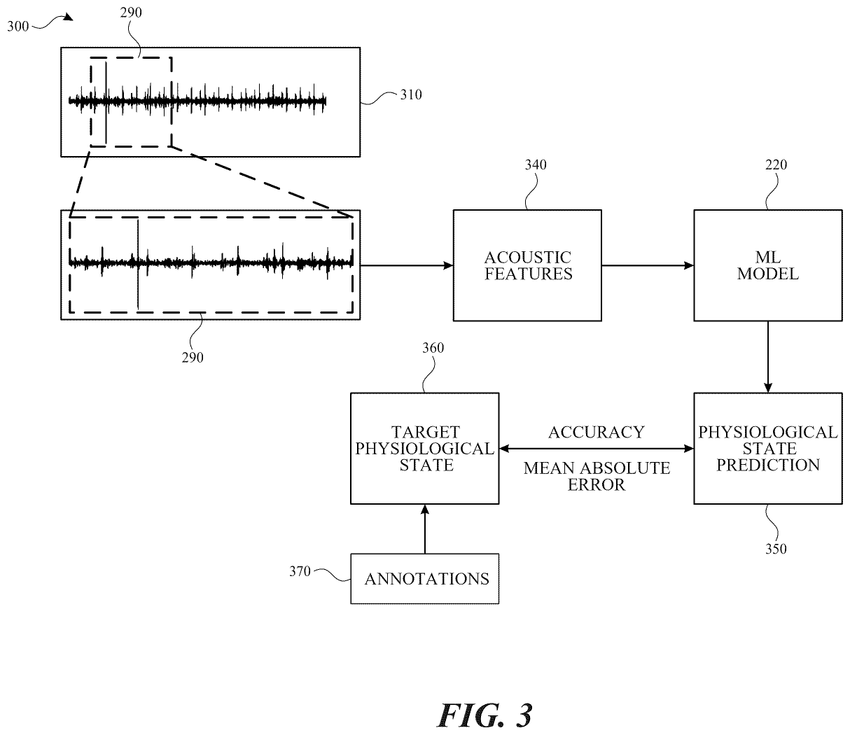

In one or more implementations, any one of the electronic devices 110-118 may include a heart rate detection system that incorporates (1) acoustic physiological signal data acquired from any one of the electronic devices 110-118 and (2) a detection algorithm as will be described in more detail in FIGS. 3-10 to provide (3) real-time monitoring and alert notifications. For example, when an underlying heart condition is detected by at least one of the electronic devices 110-130, the system generates alert notifications to user and/or emergency contacts depending on user preferences. In one or more other implementations, physiological state information can be logged in memory in a privacy-sensitive manner and trackable via an on-device application of the electronic devices 110-118.

In one or more implementations, the physiological signals may include phonocardiogram (PCG) data recorded by at least one of the electronic devices 110-118, such as the electronic device 118, electromyography data recorded by at least one of the electronic devices 110-118, such as the electronic device 114, electroencephalography data recorded by at least one of the electronic devices 110-118, such as the electronic device 118, electrocardiogra data recorded by at least one of the electronic devices 110-118, such as the electronic device 114, electrooculography data recorded by at least one of the electronic devices 110-118, such as the electronic device 118, and respiration data recorded by at least one of the electronic devices 110-118, such as the electronic device 118, among others.

In one or more implementations, the phonocardiogram data may include open-source heart rate sound datasets and may be recorded by digital stethoscopes, primarily used by medical professionals to amplify heart sounds and collect diagnostic reference points. While certain technologies incorporate digital stethoscopes for heart rate estimation, these technologies rely on sensors other than audio. Additionally, these digital stethoscopes have been employed in detecting murmurs. Heart rate sounds and ECG data may exhibit about a 97% accuracy in heart rate prediction and about an 87% accuracy in murmur prediction. Embodiments of the subject technology provide for facilitating robust heart rate prediction solely through acoustic data and methods to extract not just heart rate and its variations, but also to discern underlying cardiac conditions such as arrhythmia and murmurs.

In one or more implementations, the subject technology provides for estimating a physiological state, such as heart rate, heart rate variability (HRV), murmurs, arrhythmia, among others, based on acoustic data using a model driven methodology. For example, at least one of the electronic devices 110-130 may predict the physiological state based on at least a portion of the phonocardiogram data using a trained machine learning model. In one or more implementations, at least one of the electronic devices 110-118, such as the electronic device 118, may be utilized to analyze respiration and breathing patterns through audio data. Recognizing the interconnectedness of breathing and cardiovascular activity, the audio data, particularly from open-source heart rate sound datasets, among others, may offer insights into heart rate prediction using any one of the electronic devices 110-130 including other types of electronic devices such as digital stethoscopes.

In one or more implementations, rather than directly employing audio data from at least one of the electronic devices 110-118, such as the electronic device 118, for heart rate sensing, the subject technology provides for mapping audio data from other types of electronic devices, such as digital stethoscopes, to produce a transformation derived from the audio data that accurately predicts one or more physiological states of a subject, such as heart rate.

FIG. 2 illustrates an example computing architecture for a system providing for multimodal pretraining on physiological signals using machine learning in accordance with one or more implementations. For explanatory purposes, the computing architecture is described as being provided by an electronic device 200, such as by a processor and/or memory of the server 120, or by a processor and/or a memory of any other electronic device, such as the electronic device 110. Not all of the depicted components may be used in all implementations, however, and one or more implementations may include additional or different components than those shown in the figure. Variations in the arrangement and type of the components may be made without departing from the spirit or scope of the claims as set forth herein. Additional components, different components, or fewer components may be provided.

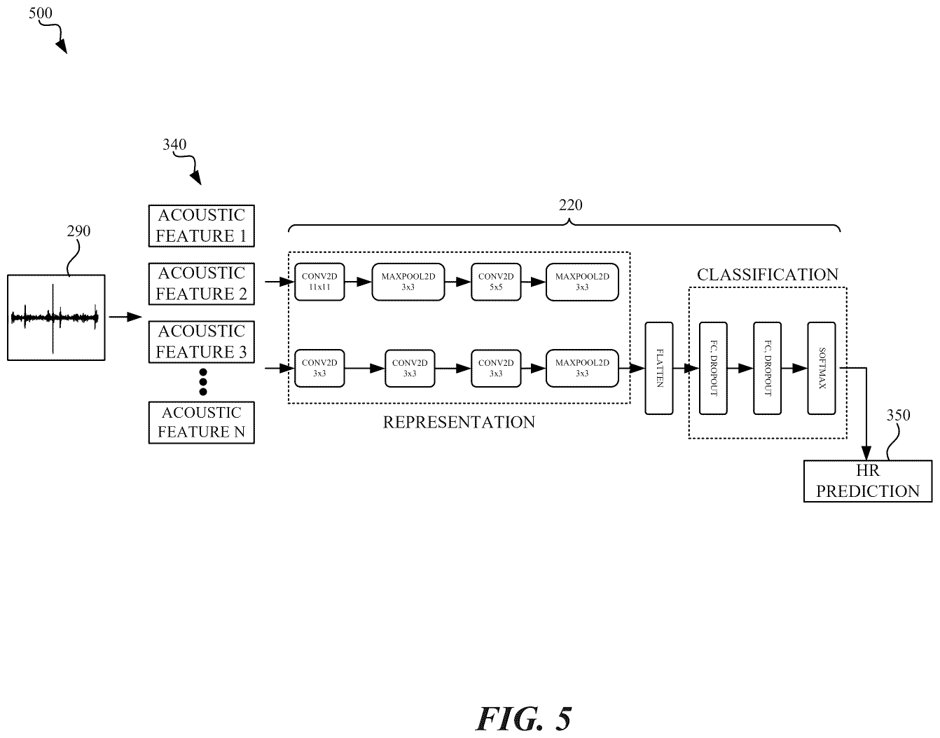

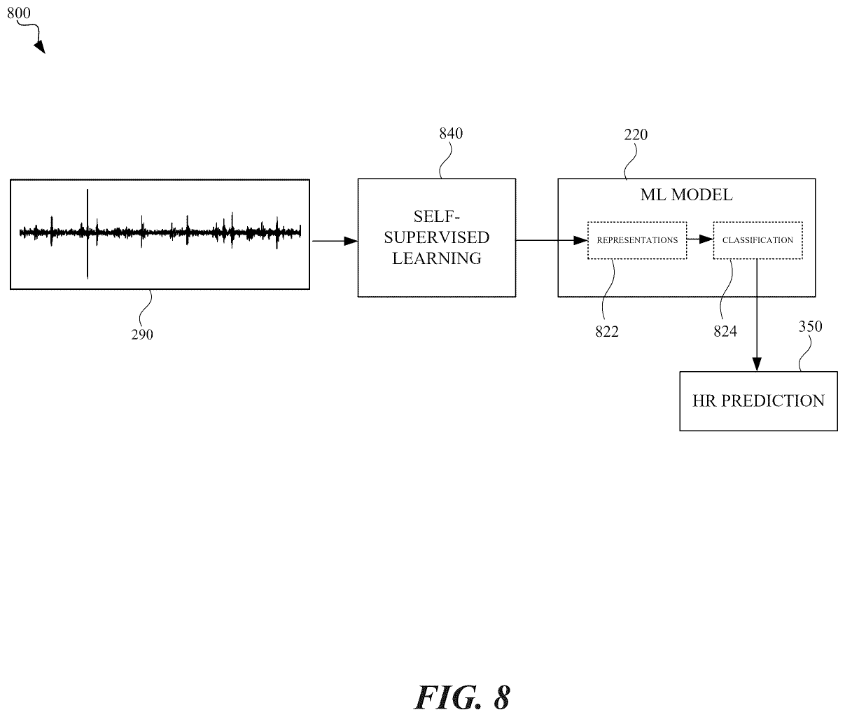

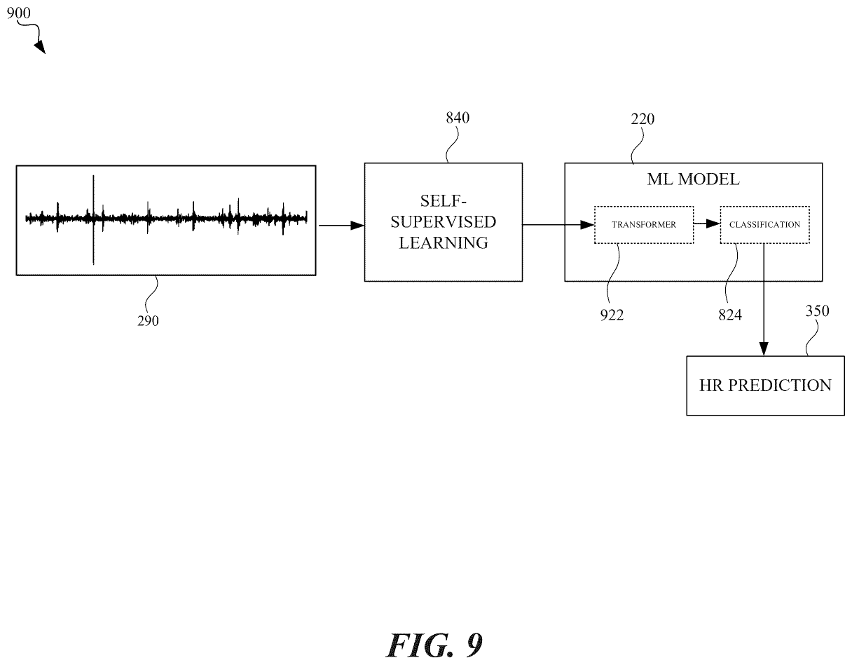

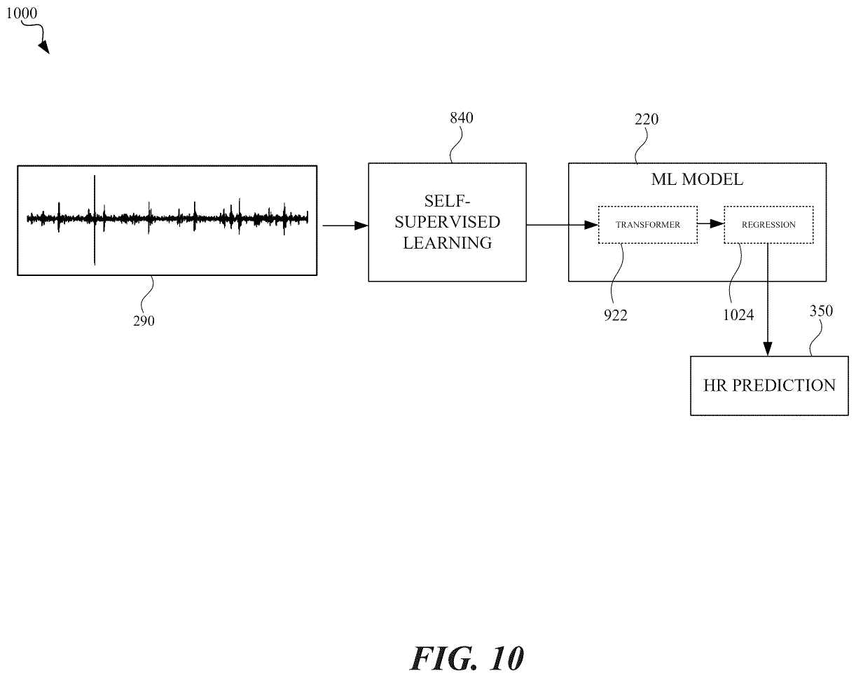

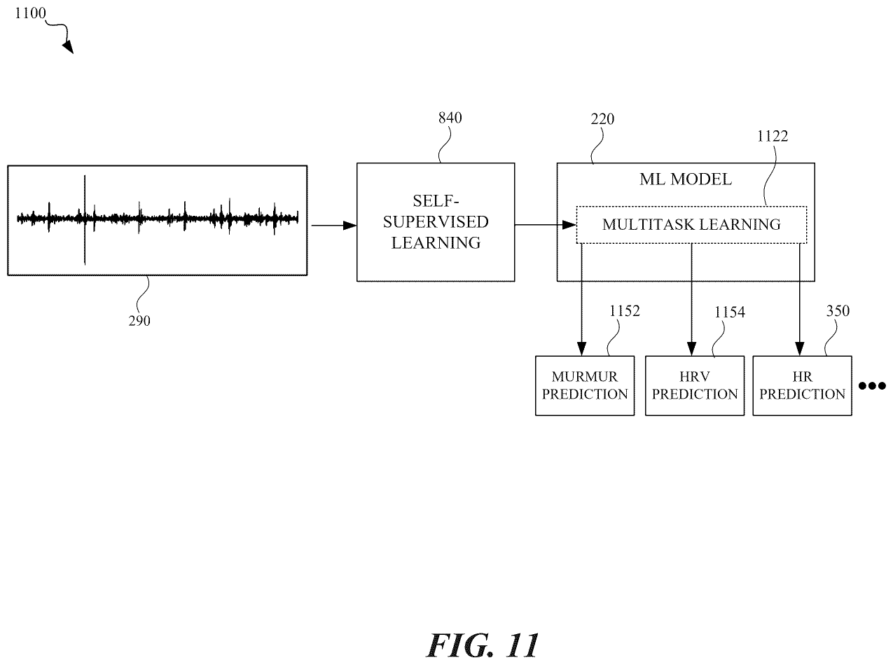

As illustrated, the electronic device 200 includes training data 210 that is stored in memory of the electronic device 200 for training a machine learning model. In an example, the server 120 may utilize one or more machine learning algorithms that uses the stored training data 210 for training a machine learning (ML) model 220. The ML model 220 may include one or more neural networks, which will be described in more detail below with reference to FIGS. 5-11. In one or more implementations, the ML model 220 includes one or more data flow paths as described with reference to FIGS. 5-7. In one or more other implementations, the ML model 220 includes a representations module and classification module as described with reference to FIG. 8. In one or more other implementations, the ML model 220 includes a transformer and classification module as described with reference to FIG. 9. In one or more other implementations, the ML model 220 includes a transformer and regression module as described with reference to FIG. 10. In one or more other implementations, the ML model 220 includes a multi-feature multitask learning module as described with reference to FIG. 11.

The training data 210 may include health-related information associated with measurable physiological signals or electrical impulses generated within a user. These signals are collected from various physiological processes in the body and carry important information about the user's health, function, and state. These physiological signals can be broadly categorized into different types, including: (1) Electrocardiogram (ECG/EKG), and (2) Phonocardiogram (PCG). In some aspects, ECG/EKG may refer to a physiological signal that measures the electrical activity of the heart. It is commonly used to assess heart rate, rhythm, and detect abnormalities in the heart's function. Training data 210 may also include demographic information (e.g., age, gender, BMI, etc.) for a user of the electronic device 110, and/or a population of other users.

The training data 210 can be used to produce a machine learning model (e.g., ML model 220) that is trained to predict heart rates, heart rate measurements, and detecting murmurs, among others, in subjects. In one or more implementations, the training data 210 includes approximately 3000 heart rate sound recordings from diverse participants, ranging from five to 80 seconds each, encompassing subjects with demographic information (e.g., varying ages, from infants to several years old). Participant profiles vary, with some presenting murmurs and others without, for example. In one or more implementations, the training data 210 may include annotations for supervised learning during training of the ML model 220. The segmentation annotations (onsets and offsets) regarding the location of fundamental heart sounds (S1 and S2), systolic period, and diastolic period obtained through a semi-supervised approach, leveraging a voting mechanism that involves one or more machine learning approaches.

In one or more other implementations, the training data 210 includes data captured via at least one of the electronic devices 110-118, such as the electronic device 118. In one or more other implementations, the training data 210 includes data captured via digital stethoscopes. In one or more other implementations, the ML model 220 is trained with digital stethoscope data to function with at least one of the electronic devices 110-118, such as the electronic device 118, to utilize its generalization capability when exposed to data from the electronic device 118, providing meaningful physiological state predictions.

In one or more implementations, challenges arise from the characteristics of PCG audio files and annotations in the training data 210, impacting the development of a robust model for heart rate prediction and murmur prediction. The audio files collected in various environments introduce diverse noises, including environmental background noises. Annotation biases and segmentation errors may further complicate attributes of the training data 210. In one or more implementations, only segments of each heart sound recording are annotated. In one or more implementations, to prepare the training data 210 with labeled data, a sliding window 250 with a predefined window length 260 and a predefined stride length 270 is applied to the raw PCG audio files (e.g., original acoustic signal 240) with an annotated period 280 longer than the predefined window length 260, as shown in FIG. 2B. As such, the electronic device 200 can generate a set of heart sound recording snippets (e.g., acoustic signal snippet 290). The average heart rate in bpm of each acoustic signal snippet 290 can be calculated by:

$$\overline{H R} = \frac{1}{N} {\sum }_{n = 1}^{n + N - 1} \frac{60}{R {R}_{n}} \qquad ( 1 )$$

In equation (1), the parameter RRₙ is the interbeat interval between the adjacent onsets of S1 waves and N is the number of S1 waves appears in the audio snippet. The appearance of heart murmur may also be assigned to each acoustic signal snippet 290 (Murmur∈{Absent, Present, Unknown}). In one or more implementations, the PCG dataset is split into the training data 210, a validation set, and a test set. The acoustic signal snippets 290 in each set can be from different subjects. In one or more other implementations, the murmur prediction applies on the acoustic signal snippets 290 with the murmur labels as Absent(0) or Present(1).

FIG. 3 conceptually illustrates an example overview 300 of physiological state prediction based on acoustic data using machine learning in accordance with one or more implementations. In one or more implementations, the illustrated pipeline includes signal extraction from audio data, subsequent extraction of acoustic features and model representations, classification to derive predicted heart rates, and comparison with target heart rates to ascertain accuracy metrics such as mean absolute errors. This pipeline can serve as a foundational structure in one or more implementations, with other pipelines receiving a broadened feature set for enhanced signal analysis and processing in one or more other implementations. This integrated methodology streamlines heart rate prediction directly from acoustic data, offering a comprehensive understanding of the model driven methodology.

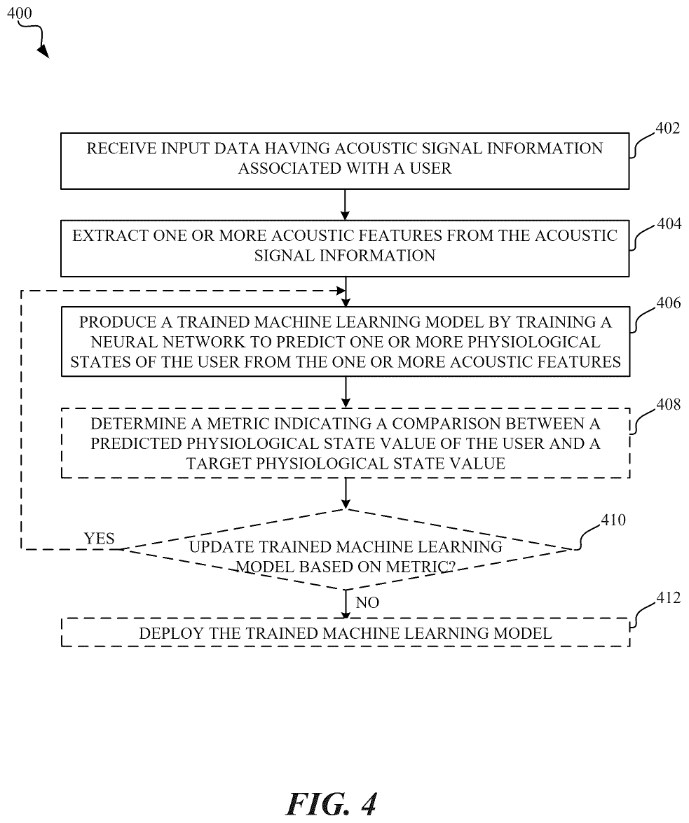

For purposes of illustration and brevity, aspects of FIG. 3 will be discussed with reference to FIG. 4. FIG. 4 is a flow chart of an example process that may be performed for frequency-aware encoding and frequency-preserving pretraining in accordance with one or more implementations. For explanatory purposes, the process 400 is primarily described herein with reference to the electronic device 110 of FIG. 1. However, the process 400 is not limited to the electronic device 110 of FIG. 1, and one or more blocks (or operations) of the process 400 may be performed by one or more other components of other suitable devices and/or servers. Further for explanatory purposes, some of the blocks of the process 400 are described herein as occurring in serial, or linearly. However, multiple blocks of the process 400 may occur in parallel. In addition, the blocks of the process 400 need not be performed in the order shown and/or one or more blocks of the process 400 need not be performed and/or can be replaced by other operations.

As illustrated in FIG. 4, at block 402, an apparatus (e.g., the electronic device 110, 112, 114, 116, 118, 120, 130) receives input data (e.g., acoustic signal snippet 290) having acoustic signal information associated with a user, such as heartbeat sounds. In the example of FIG. 2B, the acoustic signal snippet 290 is derived from the original acoustic signal 240, representing a time-series snippet of the original acoustic signal 240. In one or more implementations, the original acoustic signal 240 may include non-phonocardiogram data, revealing the prevalent presence of numerous noises. These disturbances may be encountered due to real-world dataset acquisition, encompassing movement noise, background noise, and inherent rotation biases. The labeling of data points or zero segments impacted by noise poses considerable challenges, compounded by uncertainties in heart events, notably arising from low power real-time (LPR) algorithms.

In one or more implementations, any one of the electronic devices 110-130 may process the entire original acoustic signal 240 for sampling such that the acoustic signal snippet 290 includes the original acoustic signal 240 in its entirety. In one or more other implementations, any one of the electronic devices 110-130 may process one or more segments of the original acoustic signal 240, resulting in the acoustic signal snippet 290 having a sampling length with a fixed duration.

In one or more implementations, the subject technology may include segmentation of the original acoustic signal 240 and/or the acoustic signal snippet 290. For example, any one of the electronic devices 110-130 may detect distinct cardiac events, such as systole and diastole, within the heart rate sounds (from the acoustic signal snippet 290). In one or more implementations, the subject technology also includes segmenting these unique cardiac events into respective heart rate zones. This segmentation can identify and differentiate specific cardiac events. The objective is to achieve real-time segmentation of distinct heart rate sound events that operate at various time scales from audio data, contributing to increased ML model 220 accuracy based on segmented information that captures events across different temporal scales. This integrated approach can align the most effective acoustic features with optimal segmentation, enhancing the representation and overall performance of the ML model 220.

In FIG. 3, an acoustic signal snippet 290 is received as input at an acoustic feature extraction block 340. At block 404, the apparatus extracts one or more acoustic features from the acoustic signal information. In one or more implementations, the acoustic feature extraction block 340 includes diverse signal processing technologies to extract the acoustic features from the acoustic signal snippet 290. In one or more implementations, the extraction of acoustic features encompasses several parameters, including Mel spectrograms (Mel) (N_{freq_bands},T_{frames}), Mel frequency cepstral coefficients (MFCC) (N_{freq_bands},T_{frames}), power spectral density (1,T_{frames}), and root-mean-square value (1,T_{frames}), for each frame. The power spectral density (PSD) parameter and root mean square (RMS) parameter encapsulate temporal information within each acoustic signal snippet 290, while the Mel spectrogram parameter (Mel) and Mel frequency cepstral coefficient (MFCC) parameter offer insights into both temporal and spatial information. Beyond these acoustic features, the acoustic feature extraction block 340 may perform additional signal processing, such as wavelets, given their utility in addressing time-frequency uncertainties within a fixed window length framework. In this regard, the subject technology can facilitate multi-resolution processing pipelines capable of handling varying time-frequency complexities inherent in the acoustic data.

Within the subject technology, the acoustic feature extraction block 340 can generate the acoustic features by resampling the acoustic signal snippet 290 from about 22,050 Hertz (Hz) to about 16,000 Hz. For Mel and MFCC, the acoustic feature extraction block 340 can generate about 40 Mel bands/MFCCs, with the highest frequency set to about 2,000 Hz. In one or more implementations, the window size for the short-time Fourier transform (STFT) is established at about 1,024, while the hop length is set to about 160.

In one or more other implementations, the acoustic feature extraction block 340 may perform modulation-based techniques within the signal processing of the acoustic signal snippet 290, focusing on modulation energies known for their heightened noise resilience. The acoustic feature extraction block 340 may combine multiple acoustic features to feed to the ML model 220 to bolster robustness while simultaneously enhancing noise awareness of the ML model 220. In one or more other implementations, the acoustic feature extraction block 340 may augment the data in the acoustic signal snippet 290 with noise variations, facilitating the ML model 220 to familiarize itself with diverse noise types commonly encountered in real-world scenarios.

At block 406, the apparatus produces a trained machine learning model by training a neural network to predict one or more physiological states of the user from the one or more acoustic features. In one or more implementations, the applied learning method used to train the ML model 220 involves supervised learning. The ML model 220 may operate based on cross-entropy training, employing multiple output nodes corresponding to potential heart rate values within a specified range. For example, if the heart rate range spans from 50 to 100 beats per minute, there can be respective output nodes for each integer value within this range. The ML model 220, for example, may be trained with normal heart rate value ranges for an individual of various age, gender, race, height, weight, etc. In one or more implementations, these nodes may be trained as classifiers, distinguishing between different heart rate values. In one or more other implementations, these nodes may be trained as regressors. In one or more implementations, the classifier-based approach may outperform the regressor-based approach by achieving a lower mean absolute error with the classifier approach. Its ability to discern distinctions among various heart rate events across the range, from lower to higher rates, resulted in superior performance compared to the regressor-based approach. The classifier-based approach may allow for precise identification and categorization of heart rate variations, thereby enhancing the accuracy of the ML model 220 in heart rate prediction.

The heart rate prediction may be considered a 141-class classification problem, while murmur prediction is treated as a binary classification task. The weighted cross entropy (CE) loss and binary cross-entropy (BCE) are used for heart rate (HR) prediction and heart murmur (MM) prediction tasks, respectively:

$$C {E}_{H R} = {W}_{H R} {\sum }_{b = 1}^{B} ( - log \frac{exp ( {x}_{b} , {y}_{b} )}{{\sum }_{c = 1}^{C = 141} exp ( {x}_{n , c} )} ) \qquad ( 2 )$$

$$BC {E}_{M M} = {w}_{M M} {\sum }_{a = 1}^{A} [ - {y}_{a} log {x}_{a} + ( 1 - {y}_{a} ) log ( 1 - {x}_{a} ) ] \qquad ( 3 )$$

$$ℒ = {CE}_{H R} + B C {E}_{M M} \qquad ( 4 )$$

In equation 3, A is the number of audio snippets containing heart murmur labels (Absent or Present), B is the number of acoustic signal snippets, C is the number of heart rate prediction classes, L is the training objective for MTL, and w_{HR} and w_{MM} are the weights as hyperparameters. The models are trained with a mini-batch size of about 16 for about 100 epochs. The initial learning rate can be set to be about 0.001 for one or more machine learning models.

In one or more implementations, the ML model 220 may be trained to adapt to noise present within the acoustic signal snippet 290. For example, the ML model 220 may learn to recognize and interpret heart rate sounds within noisy environments. The combination of various signal processing techniques applied to the acoustic signal snippet 290 to obtain multiple acoustic features, rather than relying on a single acoustic feature, contributes to the ML model 220 being resilient against noise. Certain representations derived from at least one of the signal processing techniques applied to the acoustic signal snippet 290, such as the MEL spectrogram, can capture the overall shape of cardiac events amidst noise interference present in the acoustic signal snippet 290, thus presenting a more robust technique compared to other signal processing techniques, such as the root mean square, which may be highly sensitive to noise.

The subject technology sets the learning objective as CE_{HR}, evaluating model performance through MAE for heart rate prediction models. The acoustic features (Mel, MFCC, PSD, and RMS) are vertically concatenated to enhance spatial information within the acoustic feature extraction block 340. As these acoustic features encompass both temporal and spatial information, the subject technology, in one or more implementations, may process these features in the temporal domain by constructing a time-convolutional neural network, long short-term memory (TCNN-LSTM) model. In one or more other implementations, the ML model 220 includes a representation module implemented as one one-dimensional (1D) convolutional layer followed by two LSTM layers with a hidden dimension of 128 for representation extraction within the representation module. In one or more other implementations, the ML model 220 also can include a classification module in succession with the representation module that is implemented as two fully connected layers incorporating dropout mechanisms to minimize overfitting and a softmax layer that are employed for heart rate prediction.

In one or more implementations, a “representation” refers to the feature maps or learned patterns obtained at different layers of the neural network. Each layer in a neural network captures certain aspects or acoustic features of the input data. In a convolutional neural network (CNN), the initial layers learn basic acoustic features. As the network progresses through deeper layers, more complex and abstract features are learned, combining the lower-level features to represent higher-level patterns relevant to the task at hand. These representations are the activations or outputs of neurons in different layers, essentially encoding information from the input data in a format that the neural network can use for classification, detection, or other tasks. The maxpooling layers can reduce the spatial dimensions of the representations by downsampling, retaining the most relevant information and discarding some details. This helps in creating a more compact and abstract representation of the input data, aiding the neural network in learning hierarchical and robust acoustic features.

Subsequently, the output from the acoustic feature extraction block 340 is directed towards a trained machine learning model (e.g., ML model 220). This ML model 220, having been previously trained on relevant data, operates on the extracted acoustic features to generate a physiological state prediction value (e.g., physiological state prediction 350).

The physiological state prediction 350 obtained from the ML model 220 serves as an indicator of a subject's physiological condition. To evaluate the accuracy of the prediction, the obtained physiological state prediction 350 is compared against one or more target physiological state values. This target value presumably represents the desired or expected physiological state. This interplay between the acoustic feature extraction block 340, the ML model 220, and the comparison process forms a comprehensive system for predicting and evaluating physiological states based on acoustic signals.

In one or more implementations, the one or more target physiological state values may be generated based on data annotations (e.g., annotations 370), outlining expected features of the target physiological state. The comparison between the actual physiological state prediction value (e.g., physiological state prediction 350) and the target physiological state value is an intermediary step in assessing the performance of the system. Deviations from the target value indicate the extent of accuracy or divergence in the prediction.

At block 408, optionally, the apparatus determines a metric indicating a comparison between a predicted physiological state value of the user (e.g., the physiological state prediction 350) and a target physiological state value (e.g., the target physiological state 360). In one or more implementations, the metrics employed for evaluating the ML model 220 performance include mean absolute error, offering an average measure of deviation from the target physiological state 360 (e.g., baseline heart rate). For example, a mean absolute error of two beats per minute implies an expected output within plus or minus two beats from the ground truth heart rate, following a standard benchmark of 10% error margin for acceptable heart rate prediction. The mean absolute error (MAE) and accuracy may be respectively defined as:

$$M A {E}_{H R} = \frac{1}{N} {\sum }_{i = 1}^{N} \vert {HR}_{p redicted , i} - H {R}_{target , i} \vert \qquad ( 5 )$$

$$A {CC}_{M M} = \frac{1}{N} {\sum }_{i = 1}^{N} \vert {MM}_{p redicted , i} - M {M}_{target , i} \vert \qquad ( 6 )$$

In one or more implementations, the evaluation of the ML model 220 may yield a mean absolute error in the range of about 1.3 to about 2.4. The evaluation of the ML model 220 based on different acoustic features may demonstrate varying performance, with the most optimal performance achieved through the combination of multiple acoustic features, yielding a mean absolute error of about 1.3. For example, different pairings of acoustic features may be integrated into distinct branches of convolutional networks within the ML model 220 to enhance acoustic feature fusion, subsequently contributing to improved performance of the ML model 220, reducing the mean absolute error to about 1.3.

At block 410, optionally, the apparatus determines whether to update the trained machine learning model based on the metric. If the apparatus determines to update the trained machine learning model based on the metric, then the process 400 proceeds to block 406 to update the trained machine learning model. For example, the apparatus updates the trained machine learning model if the metric exceeds an error threshold. Otherwise, the process 400 proceeds to block 412. At block 412, the apparatus deploys the trained machine learning model.

In one or more implementations, the ML model 220 relies on cross-entropy loss as its primary criterion. The ML model 220 also incorporates mean absolute error as an auxiliary criterion, introducing a coefficient that influences its weightage. In one or more implementation, the computed mean absolute error may be used to update the ML model 220. For example, if the MAE error becomes too large, the ML model 220 undergoes adjustments to refine its heart rate prediction. This adjustment may involve fitting the ML model 220 in a manner that reduces the MAE error. For example, when the mean absolute error reaches higher levels, the mean absolute error exerts a greater impact on weight updates of the ML model 220. In one or more implementations, operation of the ML model 220 may be a combination of cross-entropy loss and a factor multiplied by the auxiliary criterion (e.g., the mean absolute error). This integrated approach aims to fine-tune performance of the ML model 220, facilitating accurate heart rate predictions by considering both primary and auxiliary criteria.

In one or more other implementations, the mean absolute error can be reduced by a regularization process, such as multitask learning, aimed at enhancing the capacity of the ML model 220 for generalization. Increasing acoustic data collection may serve as one approach to reduce the mean absolute error, alongside making the ML model 220 more adept at handling noise. As more insights on managing uncertainties in acoustic spaces are gathered, including dealing with variations in heart rate sounds and murmurs, the subject technology can facilitate the decrease in the mean absolute error.

The subject technology may include a scheduler that facilitates ML model 220 generalization and regularization, notably improving performance by reducing the mean absolute error in both murmur prediction and heart rate prediction.

Incoming data (e.g., acoustic signal snippet 290), parametrized as acoustic features (e.g., 340), is processed through a segmentation or detection model, followed by a policy model yielding heart rate predictions. In one or more implementations, the subject technology consolidates the policy model and segmented data into a unified model (e.g., ML model 220), enabling an end-to-end process from acoustic data to direct heart rate prediction in a singular framework.

Referring back to FIG. 2, the training data 210 may include acoustic data corresponding to varied heart rates, contingent upon the sampling size of the training data 210. In one or more implementations, the mean absolute error was higher for physiological state prediction values corresponding to lower heart rates due to a smaller training data set in that range. Conversely, for physiological state prediction values corresponding to higher heart rates, the corresponding mean absolute error may not proportionately elevate compared to lower heart rates. In this regard, the distinction may lie in the nature of the acoustic data at different heart rate ranges. Lower heart rates may be associated with clinical conditions, such as underlying physiological states such as arrythmia or other physiological states necessitating a pacemaker. This segment represents a clinical spectrum. On the other end, higher heart rates may be associated with pediatric cases, often recordings from infants just a few months old. The distinction also may lie in the variability in data quality. For example, acoustic data collected from infant subjects may be meticulously obtained, potentially leading to clearer recordings. In one or more other implementations, acoustic data from adult subjects may exhibit lower clarity due to possible obstructions, such as clothing. In one or more implementations, this difference in MAE may be linked to both the clinical nature of the heart rates observed and the varying quality of the data collected across different age groups of the subjects.

FIG. 5 illustrates an example computing architecture 500 of a machine learning model for physiological state prediction based on acoustic data in accordance with one or more implementations. The traversal of a data pipeline begins with the reception of acoustic data input (e.g., the acoustic signal snippet 290), initiating a sequence of systematic operations tailored to unveil meaningful insights from the acoustic data. The initial phase includes the extraction of acoustic features 340 (e.g., acoustic feature 1, acoustic feature 2, acoustic feature 3, . . . , acoustic feature N) from the acoustic signal snippet 290, a process executed through a series of signal processing techniques, as described with reference to FIG. 3, aimed at translating the raw acoustic data into a structured array of discernible features.

The subsequent stage involves the transmission of these extracted acoustic features through a machine learning model (e.g., ML model 220 of FIG. 2) trained to decipher and interpret the information embedded within. Based on the various methods of feature extraction fed to the ML model 220, in one or more implementations, the input to the ML model 220 may include multiple feature extraction application programming interfaces (APIs). In one or more implementations, a vertical concatenation function can combine these extracted acoustic features 340, forming a stacked input to the ML model 220 for the downstream process. This function may stitch or stack the acoustic features 340 together at the feature level. In one or more implementations, the resulting input includes multiple channels, each containing different sets of acoustic features. When solely employing Mel features within the acoustic feature extraction block 340, the ML model 220 can achieve an accuracy resulting in an MAE of about 2.413 on the testing dataset. While some other feature combinations can achieve slightly improved MAE, the utilization of all four acoustic features (e.g., Mel, MFCC, PSD, RMS), alongside the LR scheduler, can result in optimal model performance.

The data pipeline may include a deep convolutional neural network, featuring multiple max-pooling layers. The subject technology can process information from both time and frequency domains by implementing a 2D-convolutional (2dCNN) model. For example, the computing architecture of the ML model 220 may include two-dimensional (2D) convolution layers and 2D maxpool layers, positioned to discern spatial hierarchies and localize patterns within the acoustic features. Specifically, within the representation module of this 2dCNN model, there are five convolutional layers, each succeeded by max-pooling layers. The convolution layers may analyze and detect structural nuances, while the maxpool layers distill and condense these representations, minimizing computational load while emphasizing the target information. This computing architecture of the ML model 220 can process the diverse acoustic features to infer heart rate predictions, employing cross-entropy as the loss function. These convolutional layers can employ various filter sizes and strides, utilizing the rectified linear unit (ReLU) activation function. The ML model 220 may then autonomously determine the relevance of these channels concerning the physiological state prediction 350, aiming to minimize cross-entropy loss in its decision-making process.

Following this, the processed representations traverse through a flattening layer, a juncture where the multidimensional structure of the acoustic features is transformed into a linear array, optimizing compatibility with subsequent layers of the ML model 220. For example, the multidimensional output from the convolutional and pooling layers undergoes transformation into a 1D vector through the flattening operation before transmission to the final classification module. The 1D vector encounters fully connected (FC) dropout layers as part of the classification module, incorporated to introduce variability and prevent overfitting during the ML model 220 training process. These FC dropout layers may selectively inhibit connections, fostering adaptability and facilitating resilience by the ML model 220 against undue reliance on specific acoustic features.

The final segment of the pipeline concludes with the softmax layer as part of the classification module, responsible for synthesizing the refined representations into predictive outputs. This softmax layer may interpret the transformed representations and generate predictions regarding physiological states (e.g., physiological state prediction 350) based on the analysis and processing of the acoustic features (e.g., 340). The performance of the 2dCNN model (MAE of 2dCNN at about 1.56) surpasses that of the TCNN-LSTM model (MAE of TCNN-LSTM at about 1.63), emphasizing the significance of integrating both temporal and spatial features within the ML model 220.

The ML model 220 determines the weighting of these acoustic features in a model-driven manner. When all these acoustic features are fed into the ML model 220, the input convolutional layers may automatically assign weights based on their utility. This weight allocation can rely on the cross-entropy loss, guiding the weighting of input streams. In one or more implementations, the weighting may be determined through a data-driven approach within the data pipeline illustrated in FIG. 5. In this regard, the ML model 220 can learn to assign significance to various acoustic features based on their relevance, optimizing its decision-making process concerning these extracted features.

The subject technology facilitates enhancing noise robustness, incorporating data augmentation techniques across various signal-to-noise ratio (SNR) ranges during the training of the ML model 220. This approach aims to bolster the resilience of the ML model 220 against acoustic distortions present in the acoustic signal snippet 290. Additionally, considerations involve refining policy-based decisions at the final output layers (e.g., softmax) to further enhance decision-making.

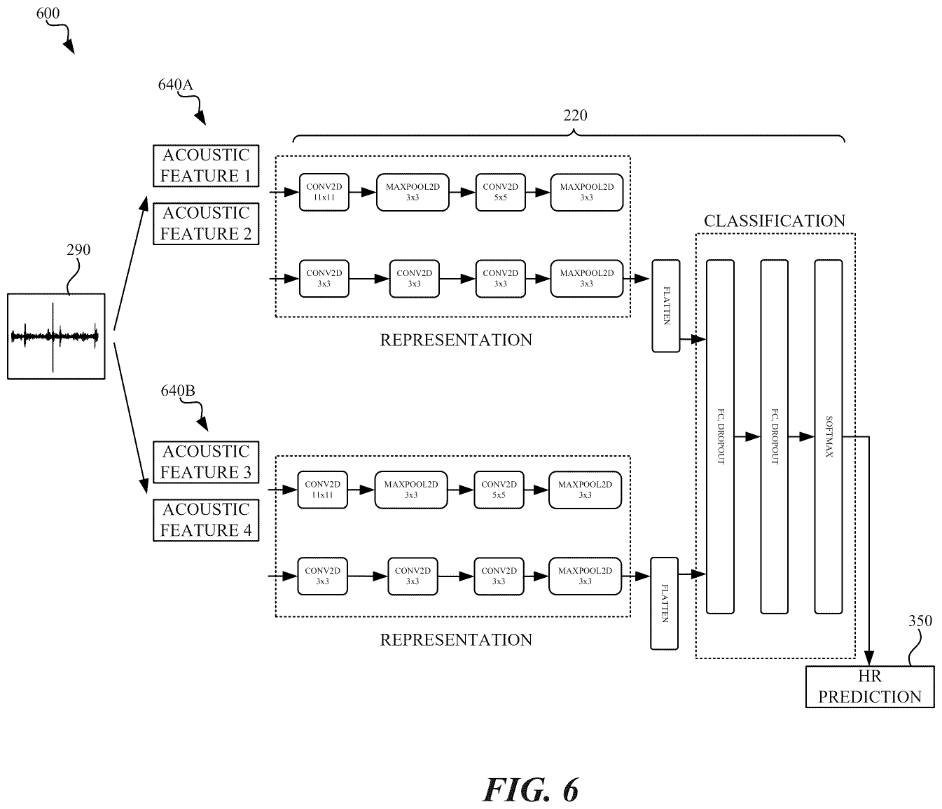

FIG. 6 illustrates another example computing architecture 600 of a machine learning model for physiological state prediction based on acoustic data in accordance with one or more implementations. The traversal of the data pipeline as illustrated in FIG. 6 begins with the reception of acoustic data input (e.g., the acoustic signal snippet 290). The initial phase includes the extraction of acoustic features 640A and 640B (e.g., acoustic feature 1, acoustic feature 2, acoustic feature 3, . . . , acoustic feature N) from the acoustic signal snippet 290, a process executed through a series of signal processing techniques, as described with reference to FIG. 3.

The subsequent stage involves the transmission of these extracted acoustic features through a machine learning model (e.g., ML model 220 of FIG. 2) trained to decipher and interpret the information embedded within. In one or more implementations, the ML model 220 may be implemented as a fusion 2D-convolutional model (2dCNN-Fusion). Within the ML model 220 as illustrated in FIG. 6, the extracted acoustic features 640A and 640B are branched into multiple neural network pathways. Each neural network branch may handle a distinct pairing of extracted acoustic features. For example, a first neural network branch may receive a first pairing of extracted acoustic features 640A that includes acoustic feature 1 and acoustic feature 2, whereas a second neural network branch may receive a second pairing of extracted acoustic features 640B that includes acoustic feature 3 and acoustic feature 4. In this 2dCNN-Fusion model, the vertical concatenations of Mel and PSD, along with MFCC and RMS, are separately fed into two representation modules, each followed by flattening operations. The resulting flattened representations are then directed to the classification module. Different combinations of these feature extractions can be applied to each neural network branch depending on implementation. Each neural network branch may include 2D convolution layers and 2D maxpool layers, positioned to discern spatial hierarchies and localize patterns within the acoustic features.

Embedded within these neural network branches are customized configurations of layers, commencing with dedicated flattening layers. These flattening layers may serve as individual conduits, transforming the multidimensional representations from their respective acoustic feature pairings into linear arrays. Following this transformation, each neural network branch may feed through a series of two FC dropout layers. These dropout layers can selectively inhibit connections during training across the diverse inputs from the multiple neural network branches. The collective outputs from these diverse branches may then be fed into a shared softmax layer, synthesizing the combination of representations from the various pathways to generate predictions concerning physiological states (e.g., 350).

The computing architecture illustrated in FIG. 6 can be configured to minimize the mean absolute error while concurrently enhancing the accuracy of murmur prediction, heart rate prediction, and other physiological state predictions. For example, the 2dCNN-Fusion model exhibits slightly improved performance (e.g., MAE of 2dCNN-Fusion at about 1.41) compared to the baseline 2dCNN model (e.g., MAE of 2dCNN at about 1.56). In one or more implementations, the 2dCNN model may be implemented with a step-wise learning rate (LR) scheduling strategy that allows it to reduce its MAE (e.g., to about 1.312). This LR scheduler can activate once the MAE associated with the validation set drops below 2.0, employing a step size of 2 and a decay rate of about 0.1.

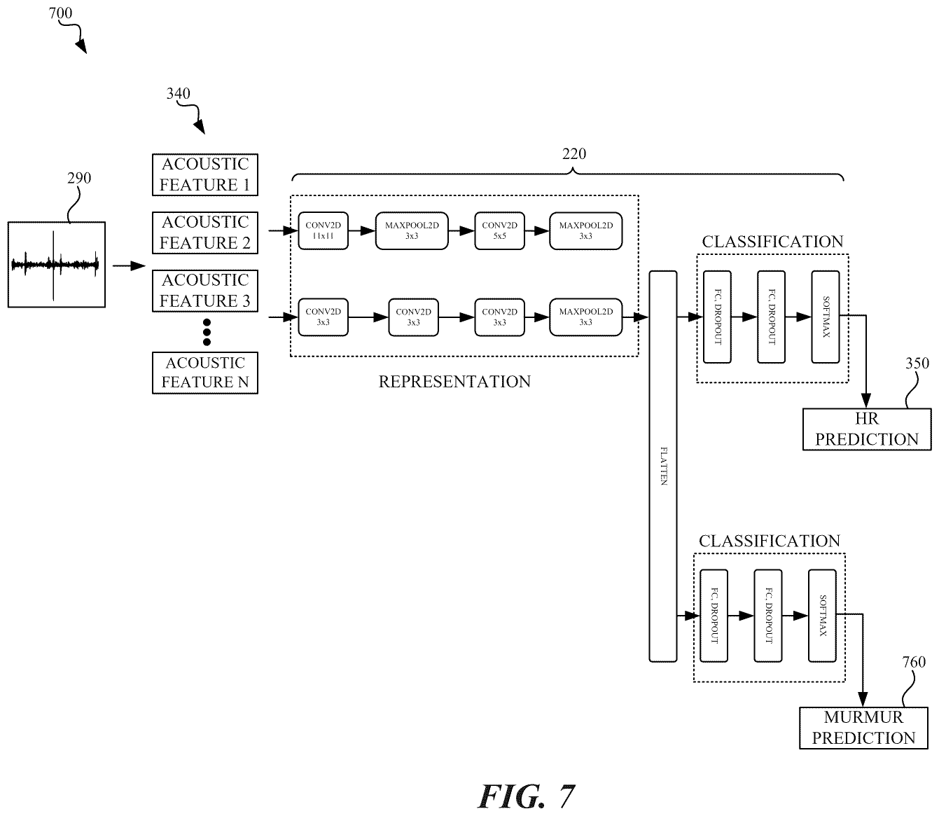

FIG. 7 illustrates yet another example computing architecture 700 of a machine learning model for physiological state prediction based on acoustic data in accordance with one or more implementations. To facilitate both heart rate prediction and heart murmur prediction within the same model framework, the subject technology includes the 2dCNN-MTL model, as depicted in FIG. 7. This model integrates an additional classification module following the flattening layer of the 2dCNN model, specifically tailored for murmur prediction. The training objective for the 2dCNN-MTL model is described by Equation (4). In one or more implementations, the ML model 220 implements various weights (a and B) in the L. In one or more other implementations, the ML model 220 incorporates an LR scheduler.

The traversal of the data pipeline as illustrated in FIG. 7 begins with the collection of acoustic data input (e.g., the acoustic signal snippet 290). The initial phase includes the extraction of acoustic features 340 (e.g., acoustic feature 1, acoustic feature 2, acoustic feature 3, . . . , acoustic feature N) from the acoustic signal snippet 290, a process executed through a series of signal processing techniques, as described with reference to FIG. 3.

The subsequent stage involves the transmission of these extracted acoustic features through a machine learning model (e.g., ML model 220 of FIG. 2) trained to decipher and interpret the information embedded within. The ML model 220 may include an initial phase that includes a single neural network branch. This neural network branch may include 2D convolution layers and 2D maxpool layers, positioned to discern spatial hierarchies and localize patterns within the acoustic features.

Following this, the processed representations traverse through a common flattening layer, a juncture where the multidimensional structure of the acoustic features is transformed into a linear array, optimizing compatibility with subsequent layers of the ML model 220. This flattening layer may harmonize the multidimensional representations into the linear array, ensuring compatibility for subsequent bifurcation into two distinct final segment pathways.

Subsequently, the linear array is branched into multiple final segment pathways to handle the transformed representations. Each pathway may encounter a series of two fully connected dropout layers, incorporated to introduce variability and prevent overfitting during the ML model 220 training process. These FC dropout layers may selectively inhibit connections at each pathway, fostering adaptability and facilitating resilience by the ML model 220 against undue reliance on specific acoustic features.

Each final segment pathway of the pipeline concludes with a separate softmax layer, responsible for synthesizing the refined representations into predictive outputs across the two pathways. In one or more implementations, the softmax layer in a first final segment pathway may interpret the transformed representations and generate predictions regarding a physiological state (e.g., physiological state prediction 350) based on the analysis and processing of the acoustic features (e.g., 340) along the first final segment pathway. In one or more other implementations, the softmax layer in a second final segment pathway may interpret the transformed representations and generate predictions regarding another physiological state (e.g., murmur prediction 760) based on the analysis and processing of the acoustic features (e.g., 340) along the second final segment pathway.

Setting both w_{HR} and w_{MM} to 1 in the MTL loss yields the best 2dCNN-MTL models for heart rate prediction, performing comparably to the 2dCNN model as described with reference to FIG. 5. The murmur prediction accuracy (ACC_{MM}) can reach about 92.39% and about 95.19% with and without the step-wise LR scheduler, respectively. For murmur prediction, the best 2dCNN-MTL models can achieve an accuracy of about 97.49%.

FIGS. 8-11 are schematic diagrams illustrating different example machine learning models in accordance with one or more implementations. Referring to FIG. 8, the schematic diagram illustrates a pipeline 800 with a first type of machine learning model. Similarly to FIG. 3, the pipeline 800 illustrated in FIG. 8 includes signal processing of audio data (e.g., snippet 290), processing of model representations, classification to derive predicted heart rates (e.g., 350), and comparison with target heart rates to ascertain accuracy metrics such as mean absolute errors.

In contrast to the computing architecture illustrated in FIG. 3, the pipeline 800 illustrated in FIG. 8 includes self-supervised learning in lieu of extraction of acoustic features from the acoustic signal snippet 290. The self-supervised learning block 840 may exemplify the utilization of pre-trained language models. Some examples of these pre-trained models include Vic-based models, which undergo training on a vast volume of audio samples, encompassing various audio events, including breathing and heart sounds captured via digital stethoscopes. The self-supervised learning block 840 may include pre-trained representations that offer a distinct acoustic feature representation based on embeddings derived from models adept in comprehending various acoustic occurrences. These pre-trained language models may possess comprehensive knowledge of diverse acoustic elements, spanning from background noise to heart and breathing sounds.

In one or more implementations, the first type of machine learning model included in the pipeline 800 includes a representations module 822 in succession with a classification module 824. The representations module 822 may operate by scanning the pre-trained representations of the acoustic signal snippet 290 from the self-supervised learning block 840 through multiple convolutional layers. These convolutional layers may extract various features at different levels of abstraction. For example, early convolutional layers may detect edges or basic shapes, while deeper layers may capture complex patterns or textures.

After the representations module 822 processes the input, it generates a rich representation of the acoustic data, which encapsulates important features. This representation is then passed to the classification module 824. The classification module 824 can be a fully connected neural network that takes these pre-trained representation features and learns to map them to specific classes or labels (e.g., heart rate prediction, heart rate variability prediction, murmur prediction, arrythmia prediction, etc.). The classification module 824 may allow for precise identification and categorization of heart rate variations, thereby enhancing the accuracy of the ML model 220 in predictions of physiological states (e.g., heart rate prediction 350).

Referring to FIG. 9, the schematic diagram illustrates a pipeline 900 with a second type of machine learning model. Similarly to FIG. 3, the pipeline 900 illustrated in FIG. 9 includes signal processing of audio data (e.g., acoustic signal snippet 290), classification to derive predicted heart rates (e.g., 350), and comparison with target heart rates to ascertain accuracy metrics such as mean absolute errors. In contrast to FIG. 8, the ML model 220 illustrated in FIG. 9 includes a transformer module 922 in succession with the classification module 824. The transformer module 922 may operate by breaking down the pre-trained representations of the acoustic signal snippet 290 from the self-supervised learning block 840 into smaller parts and analyzing their relationships. The transformer module 922 may attend to different portions of the acoustic data to understand the context and connections between the portions. The transformer module 922 may include multiple transformer layers, where each transformer layer can refine this understanding by passing information between different parts of the acoustic data through a series of self-attention mechanisms. Following the transformer module 922, the classification module 824 may receive the refined and transformed information from the transformer module 922 and makes predictions of the physiological states 350.

Referring to FIG. 10, the schematic diagram illustrates a pipeline 1000 with a third type of machine learning model. Similarly to FIG. 9, the pipeline 1000 illustrated in FIG. 10 includes signal processing of audio data (e.g., acoustic signal snippet 290), processing the pre-trained acoustic features with a transformer, and comparison with target heart rates to ascertain accuracy metrics such as mean absolute errors. In contrast to FIG. 9, the ML model 220 illustrated in FIG. 10 includes the transformer module 922 in succession with a regression module 1024.

The transformer module 922 may operate by breaking down the pre-trained representations of the acoustic signal snippet 290 from the self-supervised learning block 840 into smaller parts and analyzing their relationships. The transformer module 922 may attend to different portions of the acoustic data to understand the context and connections between the portions. The transformer module 922 may include multiple transformer layers, where each transformer layer can refine this understanding by passing information between different parts of the acoustic data through a series of self-attention mechanisms. Following the transformer module 922, the regression module 1024 may operate by predicting continuous values. The regression module 1024 takes the refined and transformed information from the transformer module 922 and maps it to a single output: the physiological state prediction 350.

Referring to FIG. 11, the schematic diagram illustrates a pipeline 1100 with a fourth type of machine learning model. Similarly to FIGS. 8-10, the pipeline 1100 illustrated in FIG. 11 includes signal processing of audio data (e.g., acoustic signal snippet 290), processing the pre-trained acoustic features with a machine learning model, and comparison with target heart rates to ascertain accuracy metrics such as mean absolute errors. In contrast to FIGS. 8-10, the ML model 220 illustrated in FIG. 11 includes a multitask learning module 1122 as described with reference to FIG. 7.

In one or more implementations, the ML model 220 employs the multitask learning module 1122 that receives pre-trained representations of the acoustic signal snippet 290 from the self-supervised learning block 840. The pre-trained representations encode meaningful information about the acoustic signal snippet 290, capturing different aspects such as frequency, pitch, and temporal patterns, among others. The multitask learning module 1122 may operate by simultaneously addressing multiple tasks. For example, the multitask learning module 1122 may have branches specialized for different task, such as heart rate prediction 350, murmur prediction 1152, heart rate variability prediction 1154, among others. Each branch may extract task-specific information from the pre-trained acoustic features. By sharing and jointly processing the pre-trained representations across multiple tasks, the multitask learning module 1122 aims to leverage the commonality and relationships present in the acoustic signal snippet 290.

FIG. 12 illustrates an electronic system 1200 with which one or more implementations of the subject technology may be implemented. The electronic system 1200 can be, and/or can be a part of, the electronic device 120, and/or the server 120 shown in FIG. 1. The electronic system 1200 may include various types of computer readable media and interfaces for various other types of computer readable media. The electronic system 1200 includes a bus 1208, one or more processing unit(s) 1212, a system memory 1204 (and/or buffer), a ROM 1210, a permanent storage device 1202, an input device interface 1214, an output device interface 1206, and one or more network interfaces 1216, or subsets and variations thereof.

The bus 1208 collectively represents all system, peripheral, and chipset buses that communicatively connect the numerous internal devices of the electronic system 1200. In one or more implementations, the bus 1208 communicatively connects the one or more processing unit(s) 1212 with the ROM 1210, the system memory 1204, and the permanent storage device 1202. From these various memory units, the one or more processing unit(s) 1212 retrieves instructions to execute and data to process in order to execute the processes of the subject disclosure. The one or more processing unit(s) 1212 can be a single processor or a multi-core processor in different implementations.

The ROM 1210 stores static data and instructions that are needed by the one or more processing unit(s) 1212 and other modules of the electronic system 1200. The permanent storage device 1202, on the other hand, may be a read-and-write memory device. The permanent storage device 1202 may be a non-volatile memory unit that stores instructions and data even when the electronic system 1200 is off. In one or more implementations, a mass-storage device (such as a magnetic or optical disk and its corresponding disk drive) may be used as the permanent storage device 1202.

In one or more implementations, a removable storage device (such as a flash drive, and its corresponding solid state drive) may be used as the permanent storage device 1202. Like the permanent storage device 1202, the system memory 1204 may be a read-and-write memory device. However, unlike the permanent storage device 1202, the system memory 1204 may be a volatile read-and-write memory, such as random access memory. The system memory 1204 may store any of the instructions and data that one or more processing unit(s) 1212 may need at runtime. In one or more implementations, the processes of the subject disclosure are stored in the system memory 1204, the permanent storage device 1202, and/or the ROM 1210. From these various memory units, the one or more processing unit(s) 1212 retrieves instructions to execute and data to process in order to execute the processes of one or more implementations.

The bus 1208 also connects to the input device interface 1214 and output device interface 1206. The input device interface 1214 enables a user to communicate information and select commands to the electronic system 1200. Input devices that may be used with the input device interface 1214 may include, for example, alphanumeric keyboards and pointing devices (also called “cursor control devices”). The output device interface 1206 may enable, for example, the display of images generated by electronic system 1200. Output devices that may be used with the output device interface 1206 may include, for example, printers and display devices, such as a liquid crystal display (LCD), a light emitting diode (LED) display, an organic light emitting diode (OLED) display, a flexible display, a flat panel display, a solid state display, a projector, or any other device for outputting information. One or more implementations may include devices that function as both input and output devices, such as a touchscreen. In these implementations, feedback provided to the user can be any form of sensory feedback, such as visual feedback, auditory feedback, or tactile feedback; and input from the user can be received in any form, including acoustic, speech, or tactile input.

Finally, as shown in FIG. 12, the bus 1208 also couples the electronic system 1200 to one or more networks and/or to one or more network nodes, such as the electronic device 120 shown in FIG. 1, through the one or more network interface(s) 1216. In this manner, the electronic system 1200 can be a part of a network of computers (such as a LAN, a wide area network (“WAN”), or an Intranet, or a network of networks, such as the Internet. Any or all components of the electronic system 1200 can be used in conjunction with the subject disclosure.

Implementations within the scope of the present disclosure can be partially or entirely realized using a tangible computer-readable storage medium (or multiple tangible computer-readable storage media of one or more types) encoding one or more instructions. The tangible computer-readable storage medium also can be non-transitory in nature.

The computer-readable storage medium can be any storage medium that can be read, written, or otherwise accessed by a general purpose or special purpose computing device, including any processing electronics and/or processing circuitry capable of executing instructions. For example, without limitation, the computer-readable medium can include any volatile semiconductor memory, such as RAM, DRAM, SRAM, T-RAM, Z-RAM, and TTRAM. The computer-readable medium also can include any non-volatile semiconductor memory, such as ROM, PROM, EPROM, EEPROM, NVRAM, flash, nvSRAM, FeRAM, FeTRAM, MRAM, PRAM, CBRAM, SONOS, RRAM, NRAM, racetrack memory, FJG, and Millipede memory.

Further, the computer-readable storage medium can include any non-semiconductor memory, such as optical disk storage, magnetic disk storage, magnetic tape, other magnetic storage devices, or any other medium capable of storing one or more instructions. In one or more implementations, the tangible computer-readable storage medium can be directly coupled to a computing device, while in other implementations, the tangible computer-readable storage medium can be indirectly coupled to a computing device, e.g., via one or more wired connections, one or more wireless connections, or any combination thereof.

Instructions can be directly executable or can be used to develop executable instructions. For example, instructions can be realized as executable or non-executable machine code or as instructions in a high-level language that can be compiled to produce executable or non-executable machine code. Further, instructions also can be realized as or can include data. Computer-executable instructions also can be organized in any format, including routines, subroutines, programs, data structures, objects, modules, applications, applets, functions, etc. As recognized by those of skill in the art, details including, but not limited to, the number, structure, sequence, and organization of instructions can vary significantly without varying the underlying logic, function, processing, and output.

While the above discussion primarily refers to microprocessor or multi-core processors that execute software, one or more implementations are performed by one or more integrated circuits, such as ASICs or FPGAs. In one or more implementations, such integrated circuits execute instructions that are stored on the circuit itself.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. Skilled artisans may implement the described functionality in varying ways for each particular application. Various components and blocks may be arranged differently (e.g., arranged in a different order, or partitioned in a different way) all without departing from the scope of the subject technology.

It is understood that any specific order or hierarchy of blocks in the processes disclosed is an illustration of example approaches. Based upon design preferences, it is understood that the specific order or hierarchy of blocks in the processes may be rearranged, or that all illustrated blocks be performed. Any of the blocks may be performed simultaneously. In one or more implementations, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

As used in this specification and any claims of this application, the terms “base station”, “receiver”, “computer”, “server”, “processor”, and “memory” all refer to electronic or other technological devices. These terms exclude people or groups of people. For the purposes of the specification, the terms “display” or “displaying” means displaying on an electronic device.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The predicate words “configured to”, “operable to”, and “programmed to” do not imply any particular tangible or intangible modification of a subject, but, rather, are intended to be used interchangeably. In one or more implementations, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

Phrases such as an aspect, the aspect, another aspect, some aspects, one or more aspects, an implementation, the implementation, another implementation, some implementations, one or more implementations, an embodiment, the embodiment, another embodiment, some implementations, one or more implementations, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

The word “exemplary” is used herein to mean “serving as an example, instance, or illustration”. Any embodiment described herein as “exemplary” or as an “example” is not necessarily to be construed as preferred or advantageous over other implementations. Furthermore, to the extent that the term “include”, “have”, or the like is used in the description or the claims, such term is intended to be inclusive in a manner similar to the term “comprise” as “comprise” is interpreted when employed as a transitional word in a claim.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for”.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but are to be accorded the full scope consistent with the language claims, wherein reference to an element in the singular is not intended to mean “one and only one” unless specifically so stated, but rather “one or more”. Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the subject disclosure.

What is claimed is:

1. A method, comprising:
receiving input data comprising acoustic signal information associated with a user;
extracting one or more acoustic features from the acoustic signal information; and
producing a trained machine learning model by training a neural network to predict one or more physiological states of the user from the one or more acoustic features.

2. The method of claim 1, further comprising deploying the trained machine learning model to predict the one or more physiological states of the user.

3. The method of claim 1, wherein the extracting comprises applying at least one of a plurality of signal processing techniques to the acoustic signal information to obtain the one or more acoustic features with at least one of a Mel spectrogram parameter, a Mel frequency cepstral coefficient parameter, a power spectral density parameter, or a root mean square parameter.

4. The method of claim 1, further comprising segmenting the acoustic signal information into a plurality of segments, wherein each of the plurality of segments corresponds to a physiological state zone indicating a specific physiological event of the user.

5. The method of claim 1, further comprising applying a combination of the one or more acoustic features into one or more branches of the neural network, wherein the trained machine learning model is produced based on the combination of the one or more acoustic features.

6. The method of claim 1, further comprising applying different pairings of the one or more acoustic features into respective branches of the neural network, wherein the trained machine learning model is produced based on the different pairings of the one or more acoustic features.

7. The method of claim 1, further comprising determining a metric indicating a comparison between a physiological state prediction and a target physiological state to evaluate the trained machine learning model.

8. The method of claim 7, further comprising:
determining whether the metric exceeds an error threshold; and
updating the trained machine learning model when the metric exceeds the error threshold.

9. A device, comprising:
a memory; and
one or more processors configured to:
receive a plurality of acoustic features extracted from acoustic signal information associated with a user;
determine, using a trained machine learning model, a representation for each of the plurality of acoustic features;
classify, using the trained machine learning model, the representation into one or more physiological state predictions;
determine a metric indicating a comparison between the one or more physiological state predictions and a target physiological state to evaluate the trained machine learning model;
determine whether the metric exceeds an error threshold; and
update the trained machine learning model when the metric exceeds the error threshold.

10. The device of claim 9, wherein the one or more processors are further configured to deploy the trained machine learning model to predict the one or more physiological states of the user.

11. The device of claim 9, wherein the one or more processors configured to extract the one or more acoustic features are further configured to apply at least one of a plurality of signal processing techniques to the acoustic signal information to obtain the one or more acoustic features with at least one of a Mel spectrogram parameter, a Mel frequency cepstral coefficient parameter, a power spectral density parameter, or a root mean square parameter.

12. The device of claim 9, wherein the one or more processors are further configured to segment the acoustic signal information into a plurality of segments, wherein each of the plurality of segments corresponds to a physiological state zone indicating a specific physiological event of the user.

13. The device of claim 9, wherein the one or more processors are further configured to apply a combination of the one or more acoustic features into one or more neural network branches of the trained machine learning model, wherein the trained machine learning model is produced based on the combination of the one or more acoustic features.

14. The device of claim 9, wherein the one or more processors are further configured to apply different pairings of the one or more acoustic features into respective neural network branches of the trained machine learning model, wherein the trained machine learning model is produced based on the different pairings of the one or more acoustic features.

15. A system comprising:
a processor; and
a memory device containing instructions, which when executed by the processor, cause the processor to perform operations comprising:
determining an acoustic signal snippet that corresponds to at least a portion of acoustic signal information associated with a user;
applying one or more signal processing algorithms to the acoustic signal snippet to determine one or more acoustic features;
producing a representation of the one or more acoustic features using a trained machine learning model; and
classifying the representation using the trained machine learning model to predict one or more physiological states of the user.

16. The system of claim 15, wherein the operations further comprise applying the one or more signal processing algorithms to the acoustic signal snippet to obtain the one or more acoustic features having at least one of a Mel spectrogram parameter, a Mel frequency cepstral coefficient parameter, a power spectral density parameter, or a root mean square parameter.

17. The system of claim 15, wherein the operations further comprise segmenting the acoustic signal snippet into a plurality of segments, wherein each of the plurality of segments corresponds to a physiological state zone indicating a specific physiological event of the user.

18. The system of claim 15, wherein the operations further comprise applying a combination of the one or more acoustic features into one or more neural network branches of the trained machine learning model, wherein the trained machine learning model is produced based on the combination of the one or more acoustic features.

19. The system of claim 15, wherein the operations further comprise applying different pairings of the one or more acoustic features into respective neural network branches of the trained machine learning model, wherein the trained machine learning model is produced based on the different pairings of the one or more acoustic features.

20. The system of claim 15, wherein the operations further comprise:
determining a metric indicating a comparison between a physiological state prediction and a target physiological state to evaluate the trained machine learning model;
determining whether the metric exceeds an error threshold; and
updating the trained machine learning model when the metric exceeds the error threshold.
